(19) United States
(12) Patent Application Publication
(10) Pub. No.: US 2022/0054541 A1
(43) Pub. Date: Feb. 24, 2022

(54) METHOD FOR TREATING INFLAMMATORY BOWEL DISEASES

(71) Applicant: NovMetaPharma Co., Ltd., Seoul (KR)

(72) Inventor: Hoe Yune JUNG, Pohang-si (KR)

(73) Assignee: NovMetaPharma Co., Ltd., Seoul (KR)

(21) Appl. No.: 17/406,626

(22) Filed: Aug. 19, 2021

Related U.S. Application Data
(60) Provisional application No. 63/068,131, filed on Aug. 20, 2020.

(51) Int. Cl. A61K 33/30; A61P 1/12

(52) U.S. Cl. CPC A61K 33/30; A61K 38/12; A61P 1/12

(57) ABSTRACT

A new use of a cyclo(His-Pro) is disclosed. In particular, a method for treating inflammatory bowel disease which includes administering an effective amount of an isolated cyclo(His-Pro) or a pharmaceutically acceptable salt thereof to a subject in need thereof is disclosed.

### CROSS REFERENCE TO RELATED APPLICATIONS

This application claims priority to and benefits based on U.S. Provisional Application No. 63/068,131 filed Aug. 20, 2020, of which the content is incorporated herein by reference in its entirety.

### TECHNICAL FIELD

The present disclosure is directed to a composition effective in treating and/or preventing inflammatory bowel disease. The present disclosure also is directed to a method for treating and/or preventing inflammatory bowel disease using the composition.

### BACKGROUND

Inflammatory Bowel Disease (IBD) is a term used to describe disorders that involve chronic inflammation of the digestive tract. IBD is generally attributed to an inappropriate immunologic response to otherwise commensal flora in a genetically susceptible host. Symptoms of IBD most commonly include diarrhea, bloody stool, abdominal pain, weight loss, and fever. See, US FDA, Center for Drug Evaluation and Research (CDER), Ulcerative Colitis: Clinical Trial Endpoints, Guidance for Industry, August 2016. Types of IBD include ulcerative colitis and Crohn's disease.

Ulcerative colitis (UC) causes long-lasting inflammation and sores (ulcers) in the innermost lining of the large intestine (colon) and rectum. UC is usually characterized by inflammation of the colon and the rectum.

Crohn's disease (CD) is characterized by inflammation of the lining of the digestive tract, which often spreads deep into affected tissues. CD commonly manifests as inflammation of the small intestine, but can affect other parts of the body as well, in a non-contiguous manner (patchy inflammation).

The exact cause of IBD is unknown, but IBD is the result of a defective immune system. The etiology of IBD appears to involve complex interactions of genetic predisposition, environmental factors, disruption of the intestinal microbiome and an overly aggressive immune response. In addition, evidence linking the ability of intestinal epithelial cells to modify the mucosal immune response, may suggest an invasive bacterial pathway. Imbalance in intestinal microbiota of gut friendly bacteria destroyed by antibiotics as well as opportunistic pathogens are implicating factors as well. Specifically, patients with IBD have been reported to have an abnormal gut microbiota. Whether this altered flora is the cause or the result of chronic inflammation remains unclear.

According to the Centers for Disease Control and Prevention (CDC), in 2015, an estimated 1.3% of US adults (3 million) reported being diagnosed with IBD (either Crohn's disease or ulcerative colitis), which indicates a large increase from 1999 (0.9% or 2 million adults).

As treatments for IBD, topical corticosteroids, antibiotics, or aminosalicylates (5-ASA) are used for mild conditions, immunomodulator or oral corticosteroids are used for moderate conditions, and biologics or surgical treatments are used for severe conditions. A list of drugs currently available for standard treatment is shown in FIG. 7.

There is still need a new effective and safe treatment for IBD.

Zinc is an essential trace element, which is absorbed in the small intestine and serves as a cofactor for numerous enzymes involved in growth, immune function, and tissue repair. Zinc levels are often low in in patients with chronic diarrhea or malabsorptive disorders. Similarly, zinc deficiency is common in patients with IBD during disease and in remission, with a prevalence ranging from 15% to 40%. Pre-clinical data as well as human studies support that zinc deficiency may contribute to mucosal inflammation in patients with IBD. In animal models, zinc deficiency exacerbates colitis and potentiates production of pro-inflammatory cytokines, including tumor necrosis factor α (TNFα). Furthermore, previous work indicates that a low zinc diet in healthy volunteers results in a decrease in the TH1 cytokines, IFN-γ and IL-2, as well as diminished lytic activity of natural killer cells. In addition to the impact of zinc on immune function, studies involving both animal models of UC and CD patients have demonstrated improvement in mucosal permeability with zinc supplementation.

Cyclo(-His-Pro) (cyclo-Hispro or CHP), C₁H₄N₄O₂, has been known as an anhydrous dipeptide having the CAS Registry Number 53109-32-3. It is an endogenous cyclic dipeptide derived in vivo from the hydrolytic removal of the amino-terminal pyroglutamic acid residue of the hypothalamic thyrotropin-releasing hormone.

Rosenthal et al. (Life Sciences 70 (2001), 337-348) reported that CHP enhanced zinc absorption and uptake by muscle tissues and by intestinal segments.

U.S. Pat. No. 5,834,032 describes the use of CHP and zinc to lower insulin levels as a method for treating insulin-resistant diabetes.

U.S. Pat. No. 10,058,520 discloses the use of thyroid hormone, CHP and a zinc salt together in treating or delaying the onset of Alzheimer's disease and/or dementia in mammals.

The present inventors surprisingly found that CHP remarkably protects intestine or intestine mucosa and improves the conditions of IBD in an animal model by reducing intestinal permeability, disease activity and reducing body weight loss.

### SUMMARY

The present disclosure provides a method for treating an inflammatory bowel disease (IBD) in a subject in need thereof by administering a composition comprising cyclo(-His-Pro) (“CHP”) to the subject.

In an embodiment, the method of treating IBD in a mammal in need thereof comprises administering an effective amount of a pharmaceutical composition comprising a CHP or a pharmaceutically acceptable salt thereof (collectively “CHP”) to the mammal. The method may further comprise administering zinc. The CHP-comprising composition and Zn-comprising composition may be administered sequentially or simultaneously. In another embodiment, CHP and zinc may be contained in a single formulation.

In an embodiment, the composition consists essentially of a CHP or a pharmaceutically acceptable salt thereof, and a pharmaceutically acceptable carrier. In an embodiment, the CHP-comprising composition does not include zinc.

According to an embodiment, the IBD includes Crohn's disease (CD) or ulcerative colitis (UC). In an embodiment, the subject with IBD is non-responsive to antibiotics, corticosteroids, and/or 5-aminosalicilic acid (5-ASA) treatments.

According to an embodiment, the cyclo(-His-Pro) or CHP is a crystalline cyclo(-His-Pro) hydrate (“CHP hydrate”). The term “CHP” or “cyclo(-His-Pro)” as used herein refers to anhydrous CHP, amorphous CHP, crystalline CHP, CHP hydrate, or a pharmaceutically acceptable salt thereof, or a combination thereof, unless specified otherwise. A CHP hydrate with improved stability is disclosed in US 2020-0017509A1 (U.S. application Ser. No. 16/448,083), of which the entire content is incorporated herein by reference.

In an embodiment, the CHP hydrate may comprise X-ray diffraction peaks in 20 values of about 13.5°-about 13.9°, about 16.8°-about 17.2°, and about 27.1°-about 27.5°.

In another embodiment, the CHP hydrate may comprise three or more X-ray diffraction peaks in 20 values selected from about 9.8°-about 10.2°, about 13.5°-about 13.9°, about 16.8°-about 17.2°, about 17.9°-about 18.3°, about 20.0°-about 20.2°, and about 27.1°-about 27.5°.

In an embodiment, the CHP hydrate is stable at typical room temperature storage conditions for about 6 months, or about 12 months, or about 18 months, or about 24 months, or about 36 months.

In another embodiment, the CHP hydrate is substantially pure. In some embodiments, the CHP hydrate material is at least about 90% pure, or at least about 95%, 96%, 97%, 98%, 99%, or 100% pure.

In another embodiment, the CHP hydrate material may be characterized by at least two of the following:

(a) an X-ray powder diffractogram comprising at least two peaks in 20 values chosen from the following list: about 9.8°-about 10.2°, about 13.5°-about 13.9°, about 16.8°-about 17.2°, about 17.9°-about 18.3°, about 20.0°-about 20.2°, and about 27.1°-about 27.5°;

(b) pKa of about 6.4;

(c) birefringent with a fragmented, rod-like morphology when analyzed by polarized light microscopy;

(d) an initial weight loss of about 6.5% (0.9 equivalent of water), followed by sample degradation at about 280° C. when analyzed by thermogravimetric analysis technique;

(e) an endotherm with an onset of about 99° C. and a peak at about 102° C. in the first heat cycle of DSC;

(f) start of dehydration below about 10% relative humidity (RH), loss of about 5.8 wt % from 10 to 0% RH (0.8 equivalent of water) and hydration from 0 to about 40% RH in the 40° C. dynamic vapor sorption analysis;

(g) start of dehydration below about 20% RH, loss of about 6.1 wt % from about 20 to 0% RH (0.8 equivalent of water) and rehydration from 0 to about 40% RH in the 50° C. dynamic vapor sorption analysis; and

(h) start of dehydration below about 20% RH, loss of about 7 wt % from about 20 to 0% RH (1.0 equivalent of water), and rehydration from 0 to about 40% RH in the 60° C. dynamic vapor sorption analysis.

In an embodiment, the composition comprising a CHP may further include zinc metal, organic or inorganic salt of zinc, zinc compound, or a zinc ion.

In an embodiment, the composition consists essentially of a CHP. According to this embodiment, the composition comprising a CHP does not include zinc metal, organic or inorganic salt of zinc, zinc compound, or a zinc ion.

In an embodiment, the compositions are a pharmaceutically acceptable composition and may comprise a pharmaceutically acceptable carriers or excipients.

In an embodiment, the individual in need of such treatment is an individual who has been diagnosed with or who is suspected of having ulcerative colitis.

In an embodiment, the effective amount of the composition comprising a CHP is administered to the individual on a schedule or regimen.

In an embodiment, the schedule or regimen is daily administration for a period of time.

In an embodiment, a method for ameliorating symptoms of inflammatory bowel disease (IBD) in a subject in need thereof includes administering a composition comprising cyclo(-His-Pro) (“CHP”) to the subject. The composition may be a pharmaceutical, health functional food, food, beverage, or dietary supplement. The composition may further comprise zinc as another active ingredient. The composition may comprise a pharmaceutically acceptable, food acceptable carrier, or other food additives.

In an embodiment, the period of time is daily for about two weeks, daily for about three weeks, daily for about four weeks, daily for about five weeks, daily for about six weeks, daily for about seven weeks, daily for about eight weeks, daily for about nine weeks, daily for about ten weeks, daily for about two months, daily for about three months, daily for about four months, daily for about five months, or daily for about six months.

In an embodiment, the effective amount of the CHP is administered until the severity of one or more symptoms associated with ulcerative colitis, specifically, a flare of ulcerative colitis, has reduced or improved. For example, such an improvement or reduction of severity of a symptom associated with ulcerative colitis may be decreased or reduced abdominal pain, fewer instances of abdominal pain, longer periods of time between instances of abdominal pain, reduced or less frequent abdominal sounds, reduced or less frequent bloody stools, longer periods of time between instances of bloody stools, less frequent or less severe diarrhea, longer periods of time between incidents of diarrhea, reduced fever, reduced or lessened rectal pain, or longer period of time between incidents of rectal pain.

In an embodiment, there is provided a method of treating ulcerative colitis (UC) in an individual who has been diagnosed with UC comprising administering to the individual an effective amount of a composition comprising a CHP until at least one symptom associated with the UC has improved or ceased.

In an embodiment, there is provided a method of treating ulcerative colitis (UC) in an individual who has been diagnosed with UC comprising administering to the individual an effective amount of a composition comprising a CHP until incidents of diarrhea have ceased.

In an embodiment, the composition is administered daily for a period of 2-5 weeks, 2-6 weeks, 3-7 weeks, 3-8 weeks, 4-9 weeks, 4-10 weeks, 5-10 weeks, 6-11 weeks, 6-12 weeks, or 3-6 months.

In an embodiment, the effective amount of a CHP may be for example in a range of about 1-10 mg/day, about 10-50 mg/day, about 50-100 mg/day, about 100-150 mg/day, about 150-200 mg/day, about 200-300 mg/day, about 300-400 mg/day, about 400-500 mg/day, about 500-600 mg/day, about 600-700 mg/day, about 700-800 mg/day, about 800-900 mg/day, about 900-1000 mg/day, about 1000-1100 mg/day, about 1100-1200 mg/day, about 1200-1300 mg/day, about 1300-1400 mg/day, about 1400-1500 mg/day, about 1500-1600 mg/day, about 1600-1700 mg/day, about 1700-1800 mg/day, about 1800-1900 mg/day, about 1900-2000 mg/day, about 2000-2100 mg/day, about 2100-2200 mg/day, about 2200-2300 mg/day, about 2300-2400 mg/day, about 2400-2500 mg/day, about 2500-2600 mg/day, about 2600-2700 mg/day, about 2700-2800 mg/day, about 2800-2900 mg/day, or about 2900-3000 mg/day, calculated in term of anhydrous CHP.

In an embodiment, the effective amount of a CHP may be for example in a range of about 0.001-0.005 mg/kg, about 0.005-0.01 mg/kg, about 0.01-0.02 mg/kg, about 0.02-0.04 mg/kg, about 0.04-0.06 mg/kg, about 0.06-0.08 mg/kg, about 0.08-1 mg/kg, about 1-5 mg/kg, about 5-6 mg/kg, about 6-7 mg/kg, about 7-8 mg/kg, about 8-10 mg/kg, about 10-15 mg/kg, about 15-20 mg/kg, about 20-25 mg/kg, about 25-30 mg/kg, about 30-35 mg/kg, about 35-40 mg/kg, about 40-45 mg/kg, about 45-50 mg/kg, about 50-100 mg/kg, about 100-150 mg/kg, about 150-200 mg/kg, about 200-300 mg/kg, about 300-400 mg/kg, about 400-500 mg/kg, about 500-600 mg/kg, about 600-700 mg/kg, about 700-800 mg/kg, about 800-900 mg/kg, about 900-1000 mg/kg, about 1000-1100 mg/kg, about 1100-1200 mg/kg, about 1200-1300 mg/kg, about 1300-1400 mg/kg, about 1400-1500 mg/kg, about 1500-1600 mg/kg, about 1600-1700 mg/kg, about 1700-1800 mg/kg, about 1800-1900 mg/kg, about 1900-2000 mg/kg, about 2000-2100 mg/kg, about 2100-2200 mg/kg, about 2200-2300 mg/kg, about 2300-2400 mg/kg, about 2400-2500 mg/kg, about 2500-2600 mg/kg, about 2600-2700 mg/kg, about 2700-2800 mg/kg, about 800-2900 mg/kg, or about 2900-3000 mg/kg, calculated in term of anhydrous CHP.

In the above ranges, the amount of CHP is expressed based on anhydrous CHP. Therefore, when a CHP hydrate is used as the CHP in the composition, the amount could be converted accordingly.

In embodiments wherein zinc is employed as an active ingredient, the amount of zinc in the composition may range from about 0.1 to about 2000 mg, about 1 to 1000 mg, about 1 to 500 mg, about 10 to about 2000 mg, about 20 to about 1000 mg, about 20 to about 500 mg, about 50 to about 2000 mg, about 50 to 1000 mg, about 50 to 800 mg, or about 50 to 500 mg, as calculated in term of zinc cation.

### BRIEF DESCRIPTION OF THE DRAWINGS

The foregoing features of embodiments will be more readily understood by reference to the following detailed description, taken with reference to the accompanying drawings, in which:

FIG. 1 shows body weight changes in dextran sulfate sodium (DSS)-induced colitis model in C57BL/6J Jms Slc male mice (8-weeks old) according to Experimental Example 1. Colitis is induced by feeding 4% DSS in water in place of drinking water for 9 days, followed by feeding drinking water for 3 days. The CRP40 group of animals were administered orally with CHP hydrate (40 mg/kg body weight/day) and the CycloZ40 group of animals were administered with a combination of CHP hydrate 40 mg/kg+Zn gluconate 70 mg/kg (total 110 mg/kg of body weight/day) starting from 3 days prior to the DSS administration and continued for 12 days until the animals were sacrificed. The CHP and CycloZ (combination of CHP and Zn) were administered daily. Body weights of the animals were measured daily. FIG. 1 shows that at day 12, the weight loss of the CRP40 group animals were the least, indicating that the CHP could be effective in treating IBD, in particular UC.

FIG. 2 shows the disease score changes in DSS-induced colitis model in C57BL/6J Jms Slc male mice according to Experimental Example 1. The results in FIG. 2 shows that at day 12, the disease score of the CRP40 group animals were the lowest, indicating that the CHP could be effective in treating IBD, in particular UC.

FIG. 3 shows the survival rate of the animals during the experimental period of 14 days, according to Experimental Example 1. All of the animals in the CRP40 group and CycloZ40 survived until the end of the experiment, whereas about 70% of the animals in the vehicle (control) group (water fed group) died during the experiment.

FIG. 4(A) and FIG. 4(B) show the gross image of colon and the colon length (mm) of the test animals in the vehicle group, CRP40 group, and CycloZ40 group. The colon of the CRP40 group animals were longest.

FIG. 5 shows intestinal permeability of the test animals in the vehicle group, CHP40 group, and CycloZ40 group. The intestinal permeability was measured by following the in vivo Assay protocol below:

Fast mice 4 hours before oral feeding and for the duration of the experiment
Feed 40 kDA FITC-Dextran (10 mg/0.25 ml/mouse) by oral gavage
(3 hours later) Collect plasma
Dilute plasma 1:2 (or more) with PBS
Transfer 50-100 microliters of supernatant to a 96-well plate
Read fluorescence intensity at Ex: 490 nm and Em: 530 nm.

The results of FIG. 5 indicates that the CRP40 group animals, which were fed with CHP daily, were remarkably protected from intestinal leakage induced by DSS.

FIG. 6(A) shows micrographs of histological sections of DSS-induced colitis in mice, stained with hematoxylin and eosin (H&E). Scale bar represents 100 μm. CRP40 and CycloZ40 group were protected from intestinal tissue damage, evident by unbroken epithelial lining, colon wall submucosa thickness and lymphatic infiltration as compared to the vehicle group.

FIG. 6(B) shows the histological scores of DSS-induced colitis in mice. The result demonstrated that CRP40 group animals were histologically more resistant to intestinal inflammation as compared to the control (vehicle) group.

FIG. 7 is a list of existing drugs used for standard treatment of inflammatory bowel syndrome.

### DETAILED DESCRIPTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents and patent applications cited herein, whether supra or infra, are hereby incorporated by reference in their entirety.

### Definitions

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection.

The term “active agent” or “drug,” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The term “treating” and “treatment” as used herein, unless otherwise indicated, mean reversing, alleviating, inhibiting the progress of, or preventing the disorder or condition to which such term applies, or one or more symptoms of such disorder or condition.

The term “IBD”, “CD” and “UC” as used herein refer to Inflammatory Bowel Disease, Crohn's Disease, and Ulcerative Colitis, respectively.

The term “therapeutically effective amount” or “effective amount,” or “effective dose,” as used herein, is the amount of the CHP or CHP hydrate or a pharmaceutically acceptable salt thereof (as calculated as anhydrous CHP) and zinc (as zinc cation) present in a composition described herein that is needed to provide a desired level of drug in the secretions and tissues of the airways and lungs, or alternatively, in the bloodstream of a subject to be treated to give an anticipated physiological response or desired biological effect when such a composition is administered by the chosen route of administration. The precise amount will depend upon numerous factors, for example the specific activity of the composition, the delivery device employed, the physical characteristics of the composition, its intended use, as well as patient considerations such as severity of the disease state, patient cooperation, etc., and can be determined by one skilled in the art based upon the information provided herein.

The term “patient” or “subject” as used herein refers to a human or other mammal. The mammal can be any type of mammal including, without limitation, a mouse, rat, dog, cat, horse, sheep, goat, cow, pig, monkey, or human. In one embodiment, “patient” refers to a human.

The term “pharmaceutically acceptable salt” refers to a salt that can be pharmaceutically used, among the substances having cations and anions coupled by electrostatic attraction. Typically, it may include metal salts, salts with organic bases, salts with inorganic acids, salts with organic acids, salts with basic or acidic amino acids or the like. Examples of the metal salts may include alkali metal salts (sodium salts, potassium salts, etc.), alkaline earth metal salts (calcium salts, magnesium salts, barium salts, etc.), aluminum salts or the like; examples of the salts with organic bases may include salts with triethylamine, pyridine, picoline, 2,6-lutidine, ethanolamine, diethanolamine, triethanolamine, cyclohexylamine, dicyclohexylamine, N,N′-dibenzylethylenediamine or the like; examples of the salts with inorganic acids may include salts with hydrochloric acid, hydrobromic acid, nitric acid, sulfuric acid, phosphoric acid, or the like; examples of the salts with organic acids may include salts with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid or the like; examples of the salts with basic amino acids may include salts with arginine, lysine, ornithine or the like; and examples of the salt with acidic amino acids include salts with aspartic acid, glutamic acid or the like.

The terms “increased,” or “increase” are used herein to generally mean an increase by a statically significant amount; in some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The term “non-response” or “non-responsive” as used herein, refers to a condition characterized by a subject not responding to the induction of a therapy (primary non-response) or a condition characterized by the subject losing response to the therapy during the treatment (secondary loss of response). In some embodiments, the therapy may include any therapeutic agent or drug therapy used in the “treating” or “treatment” of a subject, as disclosed herein.

The term “susceptible” or “susceptibility” to a disease or condition as used herein, refers to a condition of the subject characterized by an increased likelihood to develop the disease or condition, as compared to an individual who is not determined to be susceptible to the disease or condition. In some embodiments, the condition comprises non-response to antibiotics, corticosteroids and/or sulfalazine (5-aminosalicyic acid compound or 5-ASA) therapy.

### DETAILED DESCRIPTION

The current standard protocol for treating IBD includes administering topical corticosteroids, antibiotics, or aminosalicylates (5-ASA) for minor conditions; immunomodulators or oral corticosteroids for moderate conditions, and surgical operation or biologics for severe or refractory conditions. As exemplified in FIG. 7, 5-ASA includes, but is not limited to, mesalamine, sulfasalazine, and other; antibiotics used to treat mild IBD conditions include, but are not limited to, ciprofloxacin, metronidazole, and the like; corticosteroids include, but are not limited to, budesonide, hydrocortisone, prednisone, and the like.

Immunomodulators used to treat moderate IBD conditions include, but are not limited to azathioprine, cyclosporine, methotrexate, and the like.

Biologics prescribed for treating severe or refractory IBD conditions include an anti-TNF (e.g., adalimumab, certolizumab, infliximab, and the like) or anti-integrin (e.g., natalizumab).

In an embodiment, the present disclosure is directed to a method for treating IBD in a subject in need thereof, comprising administering an effective amount of a CHP. In an aspect, the subject in need of the treatment may be non-responsive or susceptible to be non-responsive to the treatments using topical corticosteroid, antibiotics, and/or 5-ASA.

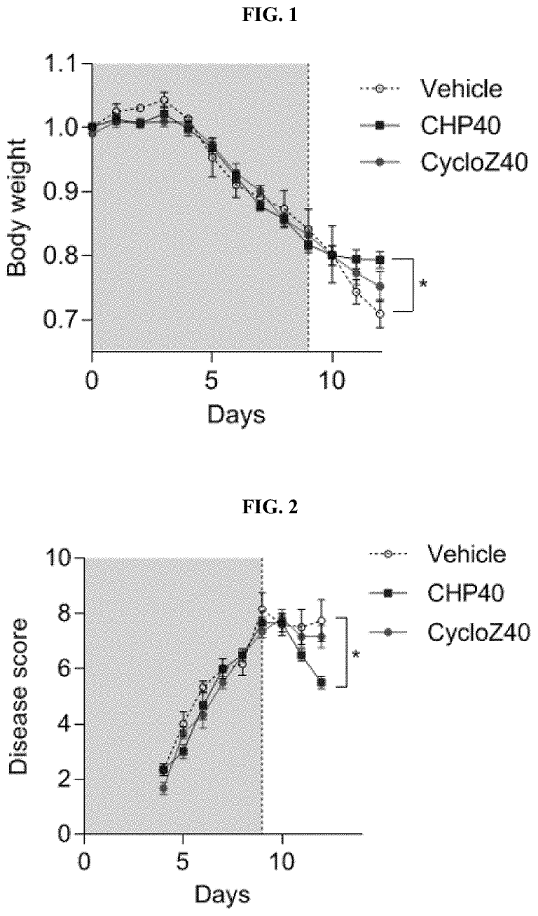

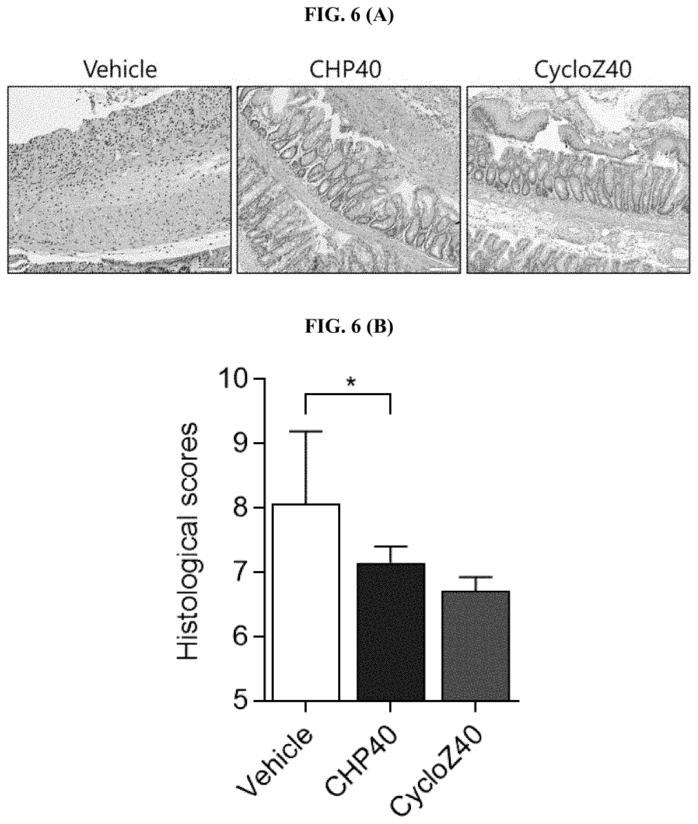

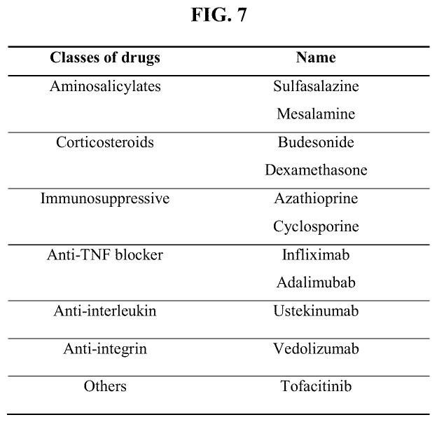

As shown in the results of Experimental Example 1 and FIGS. 1-6, it was surprisingly found that CHP shows an enhanced and remarkably excellent protection. It was known that Zn deficiency was often found in IBD patients and Zn supplementation could improve IBD treatments. CHP is reported to enhance delivery of Zn into cells. Therefore, the greater protection of intestine in DSS-induced colitis model animals administered with CHP alone at 40 mg/kg/day as a CHP monohydrate, compared to model animals administered with a combination of CHP (40 mg/kg/day as a CHP monohydrate) and Zn (70 mg/kg as zinc gluconate) were unexpected and surprising. In other experiments, results obtained from a combination of CHP and a reduced amount of Zn indicate that the combination could be more effective than CHP alone in terms of efficacy of treating IBD.

The remarkable intestine protection obtained from CHP supports the usefulness and effectiveness of CHP for treating a patient with IBD, who is not responsive to or is susceptible to non-responsive to conventional antibiotics, topical corticosteroid, and/or 5-ASA treatment(s).

In an aspect, IBD may be ulcerative colitis, necrotizing enterocolitis, collagenous colitis, lymphocytic colitis, ischaemic colitis, diversion colitis, or indeterminate colitis.

The animal model results as presented herein show protective effects of CHP alone as well as a combination of CHP and Zn. Therefore, it is stipulated that a composition comprising a CHP and optionally Zn may be used to treat IBD as well. The CHP and Zn may be included in a same composition or in separate compositions. When separate compositions comprising CHP and Zn are administered, the compositions may be administered simultaneously or sequentially.

According to an aspect of the disclosure, CHP may be anhydrous, amorphous, crystalline, or a hydrate. A CHP hydrate comprises CHP monohydrate, CHP dihydrate, CHP hemihydrate, CHP 1.5 hydrate, and the like. Therefore, the term “CHP” as used herein is intended to refer to anhydrous CHP, CHP hydrate, amorphous CHP, crystalline CHP, or a combination thereof. In some embodiments, the term “CHP” is used to collectively refer to anhydrous CHP, CHP hydrate, amorphous CHP, crystalline CHP, a pharmaceutically acceptable salt thereof, or a combination thereof.

Anhydrous amorphous cyclo (-His-Pro) (CHP) is illustrated below:

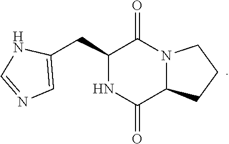 [SMILES: O=C1N[C@@H](Cc2cnc[nH]2)C(=O)N2CCC[C@@H]12]

The CHP monohydrate is illustrated below:

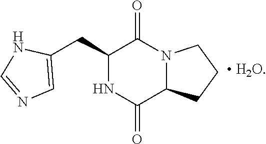 [SMILES: O=C1N[C@@H](Cc2cnc[nH]2)C(=O)N2CCC[C@@H]12]

In one embodiment, the CHP or CHP hydrate is substantially pure.

In an embodiment, the CHP hydrate is characterized by an XRPD diffractogram comprising peaks at about 17±0.2° and about 27.3±0.2° in 2θ.

One embodiment of substantially pure CHP hydrate is characterized by an X-ray powder diffractogram comprising at least three peaks chosen from the following list: 13.7, 17, 18.1, 20.2 and 27.3 degrees (±0.2° in 2θ). Another embodiment is characterized by an XRPD diffractogram comprising at least two peaks chosen from the following list: 10, 13.7, 17, 18.1, 20.2 and 27.3 degrees (±0.2° in 2θ).

A composition suitable for treating an IBD comprises a CHP as an active ingredient. The composition may comprise a pharmaceutically acceptable carrier or excipient, which is known in the art. The composition may further comprise zinc.

A composition suitable for treating an IBD may consist essentially of a CHP and may comprise a pharmaceutically acceptable carrier or excipient. The composition may be administered alone to treat IBD.

In one embodiment, a composition comprising CHP alone as an active ingredient and a composition comprising zinc may be administered separately. These compositions may be administered simultaneously or sequentially. The amount of zinc in the composition comprising zinc may range from about 0.1 to about 2000 mg, about 1 to 1000 mg, about 1 to 500 mg, about 10 to about 2000 mg, about 20 to about 1000 mg, about 20 to about 500 mg, about 50 to about 2000 mg, about 50 to 1000 mg, about 50 to 800 mg, or about 50 to 500 mg, as calculated in term of zinc cation.

In one embodiment, CHP may be present in the same composition comprising zinc or in a different composition in amount ranging from about 0.5 to about 3000 mg, from about 1 to 2000 mg, or from about 10 to about 1000 mg. In another embodiment, the amount of CHP present in the administered pharmaceutical composition may range from about 5 to about 3000 mg, from about 50 to about 2000 mg, from about 100 to about 2000 mg, from about 50 to about 1000 mg, from about 100 to about 1000 mg, from about 150 to about 2000 mg, from about 200 to about 1000 mg, from about 50 to about 800 mg, from about 100 to about 700 mg, from about 50 to about 600 mg, or from about 100 to about 1500 mg, as calculated in term of anhydrous CHP. In some embodiments, an effective amount of zinc ranges from about 0.1-1 mg/day, about 1-10 mg/day, 10-50 mg/day, 50-100 mg/day, 100-150 mg/day, 150-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, or 1900-2000 mg/day, as calculated in term of zinc cation.

In some embodiments, an effective amount of zinc ranges from about 0.1-1 mg/kg/day, about 1-10 mg/kg/day, 10-50 mg/kg/day, 50-70 mg/kg/day, 70-100 mg/kg/day, 100-120 mg/kg/day, 120-140 mg/kg/day, 140-160 mg/kg/day, 160-180 mg/kg/day, 180-200 mg/kg/day, 200-250 mg/kg/day, 250-300 mg/kg/day, or 300-400 mg/kg/day, as calculated in term of zinc cation.

In some embodiments, the weight ratio of zinc to CHP may be from about 1:10 to about 100:1 (as calculated in terms of anhydrous CHP and zinc element, unless otherwise indicated). In some embodiments, the weight ratio of zinc to CHP is from about 1:6 to about 5:1. In some embodiments, the weight ratio of zinc to CHP is from about 1:15 to about 20:1. In some embodiments, the weight ratio of zinc to CHP is from about 1:30 to about 4:1. In some embodiments, the weight ratio of zinc to CHP is from about 1:10 to about 4:1. In some embodiments, the weight ratio of zinc to CHP is from about 1:8 to about 8:1. In some embodiments, the weight ratio of zinc to CHP is from about 1:8 to about 4:1. In some embodiments, the weight ratio of zinc to CHP is from about 1:4 to about 8:1. In some embodiments, the weight ratio of zinc to CHP is from about 1:8 to about 3:1. In some embodiments, the weight ratio of zinc to CHP is from about 1:4 to about 4:1. In some embodiments, the weight ratio of zinc to CHP is from about 1:40 to about 40:1. Zinc as noted above relates to the amount of zinc cation. Zinc as noted above relates to the amount of zinc cation.

As referred to herein, numerical values for zinc represent masses or concentrations of the zinc component of a zinc salt or zinc compound. Examples of zinc salts useful in connection with the invention include zinc chloride, zinc acetate, zinc gluconate, zinc stearate, zinc sulfate, zinc oxide, zinc picolinate, zinc orotate, or zinc citrate.

Compositions intended for oral use may be prepared according to any method, and such compositions may contain one or more agents such as sweetening agents, flavoring agents, coloring agents, and/or preserving agents in order to provide pharmaceutically elegant and palatable preparations. Suitable excipients for tablets and capsules include inert diluents, such as safflower oil, lecithin, inositol, soybean shortening oil, gelatin, acacia, glycerin, titanium oxide and soybean oil. The coating of the capsules can be gelatin or a soluble polymer, as is well understood in the art. The tablets or capsules are suitable for oral administration according to a daily administration regimen.

The method of administering a composition of CHP may be accomplished by any means. For example, CHP may be administered orally, intravenously, intramuscularly, or by enema In some embodiments, it is accomplished by ingestion of a tablet, hard or soft capsules, powder, pill, drink, or lozenges. The formation of suitable oral dosage forms, may include, but are not limited to, those that are known to those having skill in the art.

In addition, the methods may also include administering the composition in single or multiple doses. Some embodiments also include administrating the ingredients of the composition in a stepwise manner, or in a manner in which one or more of the ingredients are mixed and administered prior to the administration of the other ingredients.

CHP synthesized from different biochemical sources, including histidine-proline-rich glycoprotein. High levels of CHP are present in many food sources, and are readily absorbed in the gut without chemical or enzymatic destruction.

CHP hydrate can be made by a process described in U.S. application Ser. No. 16/448,083, of which content is incorporated herein by reference, in its entirety.

CHP can be used as a substantially pure form of CHP in amorphous or crystalline form or a substantially pure form of CHP hydrate crystalline, or a mixture of CHP and CHP hydrate, as described in U.S. application Ser. No. 16/448,083, of which the entire content is incorporated herein by reference. The term “CHP” as used herein is meant to refer to anhydrous CHP, CHP hydrate, a crystalline CHP hydrate (Pattern I, Pattern II, and a mixture thereof), amorphous CHP, a pharmaceutically acceptable salt thereof, and a mixture thereof. And, the term “CHP” and “cyclo-Hispro” and “CHP/CHP hydrate” are interchangeably herein used to indicate the anhydrous CHP, CHP hydrate, a crystalline CHP hydrate (Pattern I, Pattern II, and a mixture thereof), a pharmaceutically acceptable salt thereof, or a mixture thereof, unless a specific form of CHP is intended by a specific reference to a certain form. In another embodiment, CHP is a crystalline CHP hydrate. In an embodiment, CHP hydrate is a CHP monohydrate. In some embodiment, CHP is a mixture of crystalline CHP hydrates.

In embodiments, CHP may be administered to the mammal in an amount from about 0.001 to about 3000 mg/kg. In some embodiments, the effective amount of the CHP may be about 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-6 mg/kg, 6-7 mg/kg, 7-8 mg/kg, 8-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 50-100 mg/kg, 100-150 mg/kg, 150-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg, or 2900-3000 mg/kg. The amounts are based on the amount of anhydrous CHP.

In embodiments, CHP may be administered to the mammal in an amount from about 1 to about 3000 mg/day. In some embodiments, the effective amount of the CHP may be in a range of about 1-10 mg/day, 10-50 mg/day, 50-100 mg/day, 100-150 mg/day, 150-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day, or 2900-3000 mg/day. The amounts are based on the amount of anhydrous CHP.

Certain aspects of the invention can be described in greater detail in the non-limiting Examples that follows. The following examples are included to demonstrate certain embodiments of the present disclosure. Those of skill in the art should, however, in light of the present disclosure, appreciate that modifications can be made in the specific embodiments that are disclosed and still obtain a like or similar result without departing from the spirit and scope of the intention. Therefore, all matter set forth is to be interpreted as illustrative and not in a limiting sense.

### Experimental Example 1

(1) Establishment of DSS-Induced Colitis Model Mice

Male C57BL/6J Jms Slc mice (8-week old) were fed with 4% dextran sulfate sodium (DSS) in water in place of drinking water for 9 days, followed by feeding drinking water for 3 days, inducing colitis in the mice. Mice were divided into three groups: (1) CRP40 group administered orally with CHP monohydrate (40 mg/kg body weight/day); (2) CycloZ40 group administered orally with a combination of CHP monohydrate 40 mg/kg+Zn gluconate 70 mg/kg; and (3) vehicle (control) group fed with drinking water. CHP hydrate and combination of CHP hydrate+Zn gluconate were administered to the animals starting from 3 days prior to the DSS administration and continued for 12 days until the animals were sacrificed. The above amount of the CHP monohydrate and the combination of CHP monohydrate and Zn gluconate were administered daily.

(2) Bodyweight Changes

Bodyweights of the animals were measured daily until the last date of the experiment and the results are shown in FIG. 1. FIG. 1 shows that at day 12, the weight loss of the CRP40 group animals were the least, indicating that the CHP administration to the colitis-induced animals ameliorate the bodyweight loss symptom.

(3) Evaluation of Disease Activity (Disease Score)

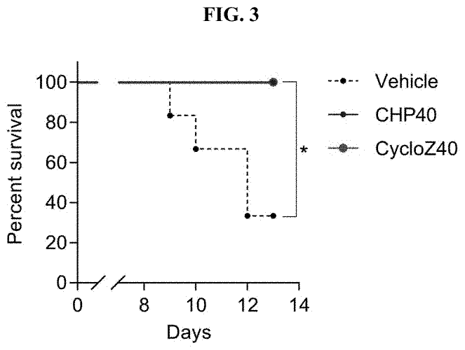

By following the Disease activity score guidance shown in Table 1 based on weight loss, stool consistency, and blood occult (Source: Stefan Wirtz et al., Chemically induced mouse models of acute and chronic intestinal inflammation Nature Protocols, Vol. 12, pages 1295-1309 (2017), doi:10.1038/nprot.2017.044.), disease scores were determined daily and the results are shown in FIG. 2.

TABLE 1

| Score | Weight loss | Stool consistency | Blood |
|---|---|---|---|
| 0 | None | Normal | Negative bemocult |
| 1 | 1-5% | Soft but still | Negative hemocult |
| | | formed | |
| 2 | 6-10% | Soft | Positive hemocult |
| 3 | 11-18% | Very soft; wet | Blood traces in stool visible |
| 4 | >18% | Watery diarrhea | Gross rectal bleeding |

The results in FIG. 2 shows that at day 12, the conditions of the CRP40 group animals were greatly improved compared to the ChocloZ40 and the vehicle groups. The results indicate that the CHP could be effective in treating IBD, in particular UC.

(4) Survival Rate

The survival rate of the animals during the experimental period of 14 days were shown in FIG. 3. All of the animals in the CRP40 group and CycloZ40 group survived until the end of the experiment, whereas about 70% of the animals in the vehicle (control) group (water fed group) died during the experiment.

(5) Colon Length

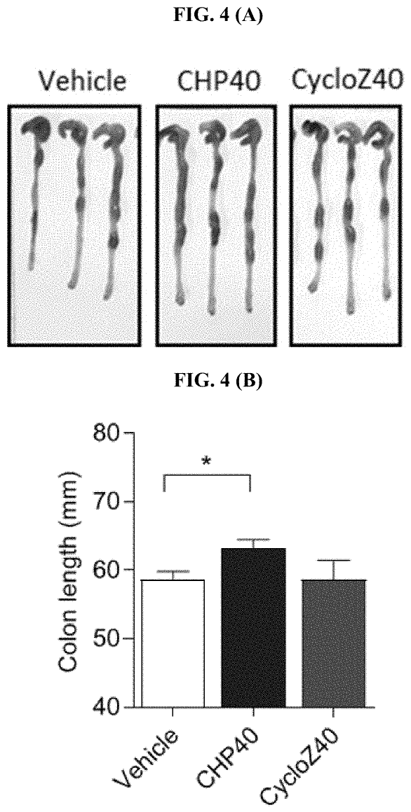

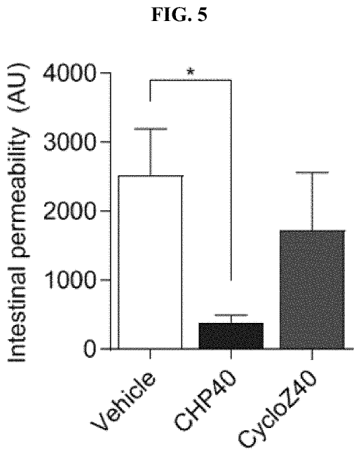

At day 12, the animals were sacrificed and their colon lengths were measured. The results are shown in FIGS. 4(A) and 4(B). The colon of the CRP40 group animals were longest, indicating that the CHP administration to the DSS-induced colitis model animal protects the intestine from colitis.

(6) Intestinal Permeability

The intestinal permeability was measured by following the in vivo Assay protocol below:
Fast mice 4 hours before oral feeding and for the duration of the experiment
Feed 40 kDa FITC-Dextran (10 mg/0.25 ml/mouse) by oral gavage
(3 hours later) Collect plasma
Dilute plasma 1:2 (or more) with PBS
Transfer 50-100 microliters of supernatant to a 96-well plate
Read fluorescence intensity at Ex: 490 nm and Em: 530 nm.

The results are shown in FIG. 5. As can be seen from the results of FIG. 5, the intestinal permeability of the CRP40 group was remarkably decreased compared to the vehicle and CycloZ40 group, indicating that the administration of CHP to the DSS-induced colitis model animal repairs the leakage or prevent the leakage of intestine. These results indicate that CHP can be used to treat or prevent an IBD, in particular UC.

(7) Histological Examination

The intestinal tissues were fixed, paraffin-embedded, and stained with hematoxylin and eosin. CHP40 and CycloZ40 group were protected from intestinal tissue damage, evident by unbroken epithelial lining, colon wall submucosa thickness and lymphatic infiltration as compared to the vehicle group.

(8) Evaluation of Histopathological Scores

Histopathological evaluation of colon was scored according to the grading system listed below.

TABLE 2
Scoring system for calculating a histological score

| | Loss of | Lymphocytes | Epithelial | Elongation of |
|---|---|---|---|---|
| Score | goblet cells | infiltration | erosion | colonic mucosa |
| 0 | None | None | None | None |
| 1 | Mild/focal | A few | Mild/focal | Mild (≥150%) |
| 2 | Numerous and | Moderate/ | Evident/ | Evident (≥150%) |
| | diffuse | focal | diffuse | |
| 3 | Numerous and | Numerous and | Evident/ | Evident (≥150%) |
| | diffuse | diffuse | diffuse | |

The result shows that CHP, optionally with zinc, can be used to treat or prevent an IBD, in particular UC.

1. A method for treating and/or preventing inflammatory bowel disease in a subject in need thereof, comprising administering an effective amount of a composition comprising a cyclo(His-Pro) or a pharmaceutically acceptable salt thereof to the subject.

2. The method of claim 1, wherein the cyclo(His-Pro) is anhydrous cyclo(His-Pro), amorphous cyclo(His-Pro), crystalline cyclo(His-Pro) hydrate, amorphous cyclo(His-Pro) hydrate, or a combination thereof.

3. The method of claim 1, wherein the crystalline cyclo(His-Pro) hydrate is a crystalline cyclo(His-Pro) monohydrate.

4. The method of claim 3, the crystalline cyclo(His-Pro) hydrate is characterized by an X-ray powder diffraction (XRPD) diffractogram comprising peaks at 20 values of 13.7°±0.2°, 17°±0.2°, and 27.3°±0.2°

5. The method of claim 4, wherein the XRPD diffractogram further comprises a peak at 20 value of 10°±0.2°.

6. The method of claim 1, wherein the composition is a pharmaceutical composition and comprises a pharmaceutically acceptable carrier.

7. The method of claim 1, wherein the composition further comprises zinc.

8. The method of claim 7, wherein the zinc is zinc ion, zinc metal, or a zinc salt selected from the group consisting of zinc chloride, zinc acetate, zinc gluconate, zinc stearate, zinc sulfate, zinc oxide, zinc picolinate, zinc orotate, and zinc citrate.

9. The method of claim 1, which further comprises administering a zinc.

10. The method of claim 9, wherein the zinc is zinc ion, zinc metal, or a zinc salt selected from the group consisting of zinc chloride, zinc acetate, zinc gluconate, zinc stearate, zinc sulfate, zinc oxide, zinc picolinate, zinc orotate, and zinc citrate.

11. The method of claim 1, wherein the subject is non-responsive to treatments by topical corticosteroid, 5-ASA, and/or antibiotics.

12. The method of claim 1, wherein the IBD is ulcerative colitis.

13. The method of claim 1, wherein the effective amount of the cyclo(His-Pro) is in a range of about 1-10 mg/day, 10-50 mg/day, 50-100 mg/day, 100-150 mg/day, 150-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day, or 2900-3000 mg/day, in term of the weight of the anhydrous cyclo(His-Pro).

14. The method of claim 1, wherein the effective amount of the cyclo(His-Pro) is in a range of about 0.001-0.005 mg/kg/day, 0.005-0.01 mg/kg/day, 0.01-0.02 mg/kg/day, 0.02-0.04 mg/kg/day, 0.04-0.06 mg/kg/day, 0.06-0.08 mg/kg/day, 0.08-1 mg/kg/day, 1-5 mg/kg/day, 5-6 mg/kg/day, 6-7 mg/kg/day, 7-8 mg/kg/day, 8-10 mg/kg/day, 10-15 mg/kg/day, 15-20 mg/kg/day, 20-25 mg/kg/day, 25-30 mg/kg/day, 30-35 mg/kg/day, 35-40 mg/kg/day, 40-45 mg/kg/day, 45-50 mg/kg/day, 50-100 mg/kg/day, 100-150 mg/kg/day, 150-200 mg/kg/day, 200-300 mg/kg/day, 300-400 mg/kg/day, 400-500 mg/kg/day, 500-600 mg/kg/day, 600-700 mg/kg/day, 700-800 mg/kg/day, 800-900 mg/kg/day, 900-1000 mg/kg/day, 1000-1100 mg/kg/day, 1100-1200 mg/kg/day, 1200-1300 mg/kg/day, 1300-1400 mg/kg/day, 1400-1500 mg/kg/day, 1500-1600 mg/kg/day, 1600-1700 mg/kg/day, 1700-1800 mg/kg/day, 1800-1900 mg/kg/day, or 1900-2000 mg/kg/day, in term of the weight of the anhydrous cyclo(His-Pro).

15. The method of claim 9, wherein the zinc is administered to the subject in an amount ranging from range of about 0.1-1 mg/day, about 1-10 mg/day, 10-50 mg/day, 50-100 mg/day, 100-150 mg/day, 150-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, or 1900-2000 mg/day, as calculated in term of zinc cation.

16. The method of claim 1, wherein the composition consists essentially of cyclo(His-Pro).

17. The method of claim 7, wherein the subject is non-responsive to treatments by topical corticosteroid, 5-ASA, and/or antibiotics.

18. The method of claim 9, wherein the subject is non-responsive to treatments by topical corticosteroid, 5-ASA, and/or antibiotics.
